(19) United States
(12) Patent Application Publication
(10) Pub. No.: US 2024/0207571 A1
(43) Pub. Date: Jun. 27, 2024

(54) SYSTEMS AND METHODS FOR CONTROLLING FLOW OF THERAPEUTIC AGENT DELIVERED TO AN INFUSION CATHETER

(71) Applicant: TriSalus Life Sciences, Inc., Westminster, CO (US)

(72) Inventors: David Benjamin Jaroch, Arvada, CO (US); Michael Brick Markham, Boulder, CO (US)

(73) Assignee: TriSalus Life Sciences, Inc., Westminster, CO (US)

(21) Appl. No.: 18/087,767

(22) Filed: Dec. 22, 2022

(51) Int. Cl. A61M 25/00; A61M 5/168

(52) U.S. Cl. CPC A61M 25/0068; A61M 5/16877

(57) ABSTRACT

Devices and systems and method for treating a patient are described herein that are used with or include at least one infusion catheter having a proximal end with a hub, a distal end with a distal tip, and a lumen extending from the proximal end to the distal tip. The devices include a connector body having a first connector for fluidly coupling and detachably connecting to the hub of the at least one infusion catheter, a second connector for fluidly coupling and detachably connecting to at least one manual syringe pump, and a passive pressure-controlled mechanical flow regulator that regulates fluid flow supplied by manual pump action of the least one manual syringe pump to the infusion catheter and delivered by the infusion catheter into the vascular system of the patient. The mechanical flow regulator can be configured to deliver a therapeutic agent or a secondary fluid supplied under pressure by pumping action of the least one manual syringe pump into and through the lumen of the infusion catheter at a desired rate of infusion.

### BACKGROUND

### 1. Field

The present disclosure relates generally to systems and methods that deliver therapeutic agents into the vascular system of a patient for treating disease of a target organ.

### 2. State of the Art

Infusion catheters are used to deliver therapeutic agents into the vascular system of a patient for treating disease of a target organ. Infusion catheters have a hub for connecting a source of the therapeutic agent.

For situations requiring infusion under pressure and/or at a controlled flow rate, current systems typically use an electrically-controlled pump to provide consistent flow for effective infusion. The operation of the electrically-controlled pump is managed by a control system that is configured to automatically control and regulate the flow of the therapeutic agent through the infusion catheter. The use of the electrically-controlled pump complicates the procedure, requiring tubing extensions and personnel to handle the therapeutic agent outside the sterile field.

### SUMMARY

Devices and systems for treating a patient are described herein that are used with or include at least one infusion catheter having a proximal end with a hub, a distal end with a distal tip, and a lumen extending from the proximal end to the distal tip. The devices include a connector body having a first connector for fluidly coupling and detachably connecting to the hub of the at least one infusion catheter, a second connector for fluidly coupling and detachably connecting to at least one manual syringe pump, and a passive pressure-controlled mechanical flow regulator that regulates fluid flow supplied by manual pump action of the least one manual syringe pump to the infusion catheter and delivered by the infusion catheter into the vascular system of the patient.

In embodiments, the passive pressure-controlled mechanical flow regulator can be configured to deliver a therapeutic agent or a secondary fluid supplied under pressure by pumping action of the least one manual syringe pump into and through the lumen of the infusion catheter at a desired rate of infusion.

In embodiments, the connector body can further include a first flowpath that extends through the first connector and is fluidly coupled to the lumen of the infusion catheter during use, and a second flowpath that extends through the second connector and is fluidly coupled to the least one manual syringe pump during use. The passive pressure-controlled mechanical flow regulator can be fluidly coupled to both the first flowpath and the second flowpath.

In embodiments, the connector body can further include a third connector for fluidly coupling and detachably connecting to at least one additional manual syringe pump, and a third flowpath that extends through the third connector and is fluidly coupled to the least one additional manual syringe pump during use. The passive pressure-controlled mechanical flow regulator can be fluidly coupled to the third flowpath.

In embodiments, the passive pressure-controlled mechanical flow regulator can include:
i) a first pressure relief valve having an inlet fluidly coupled to the second flowpath and an outlet fluidly coupled to the first flowpath;
ii) a second pressure relief valve having an inlet fluidly coupled to the first flowpath and an outlet fluidly coupled to the third flowpath;
iii) a third pressure relief valve having an inlet fluidly coupled to the third flowpath and an outlet fluidly coupled to the first flowpath; and
iv) a fourth pressure relief valve having an inlet fluidly coupled to the first flowpath and an outlet fluidly coupled to the second flowpath.

The first and third pressure relief valves can be configured to open at respective first predefined supply pressures corresponding to a desired rate of infusion, and the second and fourth pressure relief valves can be configured to open at respective second predefined supply pressures greater than the first predefined supply pressures.

In embodiments, the first and second pressure relief valves can be configured to deliver a therapeutic agent supplied under pressure by pumping action of the least one manual syringe pump into and through the lumen of the infusion catheter at the desired rate of infusion and direct any excess flow into the least one additional manual syringe pump, and the third and fourth pressure relief valves can be configured to deliver a therapeutic agent supplied under pressure by pumping action of the least one additional manual syringe pump into and through the lumen of the infusion catheter at the desired rate of infusion and direct any excess flow into the least one manual syringe pump.

In embodiments, the connector body can further include a passive flow restrictor coupled to the flowpath downstream of the passive pressure-controlled mechanical flow regulator. The passive flow restrictor can employ a fixed-size orifice or plug or other suitable passive flow regulating mechanism. The passive flow restrictor can be configured such that slow flow rates of fluid through the connector body generate enough pressure to activate the first and second pressure relief valves.

In embodiments, the connector body further can include a secondary connector for fluidly coupling and detachably connecting to at least one further manual syringe pump in a configuration that bypasses the passive pressure-controlled mechanical flow regulator.

In embodiments, the connector body can further include a first flowpath that extends through the first connector and is fluidly coupled to the lumen of the infusion catheter during use, a second flowpath that extends through the second connector and is fluidly coupled to the least one manual syringe pump during use, and an additional flowpath that extends through the secondary connector and is fluidly coupled to the least one further manual syringe pump during use. The passive pressure-controlled mechanical flow regulator can be fluidly coupled to both the first flowpath and the second flowpath, and the additional flowpath can be fluidly coupled to the second flowpath downstream of the passive pressure-controlled mechanical flow regulator.

In embodiments, the connector body can further include a check valve disposed between the passive pressure-controlled mechanical flow regulator and the additional flowpath.

In embodiments, the connector body can be configured to deliver a therapeutic agent supplied under pressure by pumping action of the least one manual syringe pump into and through the lumen of the infusion catheter at a desired rate of infusion. The connector body can be further configured to deliver a secondary fluid supplied under pressure by pumping action of the least one further manual syringe pump into and through the lumen of the infusion catheter.

In embodiments, the passive pressure-controlled mechanical flow regulator can include a chamber with an inlet port leading into interior space of the chamber and a restrictor tube that extends into the interior space of the chamber, wherein the restrictor tube includes a restrictor inlet disposed within the interior space of the chamber and an annular elastomeric membrane spaced from the restrictor inlet.

In embodiments, the elastomeric membrane can be configured to deform or deflect radially inward to regulate fluid flow through the restrictor tube.

In embodiments, the restrictor tube can further include a bypass valve having an inlet in fluid communication with the interior space of the chamber and an outlet in fluid communication with the lumen of the restrictor tube. The bypass valve can be configured to open at a predefined pressure within the interior space of the chamber that is greater than pressure corresponding to a desired infusion rate of fluid flow through the restrictor tube.

In embodiments, the passive pressure-controlled mechanical flow regulator can include a restrictor orifice of fixed size that corresponding to a desired infusion rate.

In embodiments, the fixed size of the restrictor orifice can be based on infusion of fluid of known viscosity pumped by a manual syringe pump of predefined size within a predefined operating pressure range.

Related systems and kits and methods are also described and claimed.

### BRIEF DESCRIPTION OF THE DRAWINGS

FIG. 1A is a schematic illustration of a medical system according to a first embodiment of the present disclosure.

FIG. 1B is a schematic illustration of the connector body of the system of FIG. 1A.

FIG. 1C illustrates an exemplary coil spring valves.

FIGS. 1D-1F illustrate exemplary slit valves.

FIG. 1G illustrates exemplary elastomeric duck bill valves.

FIG. 2 is a flowchart illustrating a medical procedure that uses the system of FIG. 1A to treat a patient.

FIG. 3 is a schematic illustration of an exemplary infusion catheter.

FIG. 4 is a schematic illustration of a medical system according to a second embodiment of the present disclosure.

FIG. 5 is a schematic illustration of a medical system according to a third embodiment of the present disclosure.

FIG. 6A is a schematic illustration of an exemplary pressure-controlled mechanical flow regulator that can be part of the connector body of the system of FIG. 5.

FIGS. 6B and 6C are schematic illustrations of different configurations of the annular elastomeric (flexible) membrane of the exemplary pressure-controlled mechanical flow regulator of FIG. 6A.

FIG. 7 is a schematic illustration of a medical system according to a fourth third embodiment of the present disclosure.

### DETAILED DESCRIPTION OF THE PREFERRED EMBODIMENTS

With reference to the following description, the terms “proximal” and “distal” are defined in reference to the hand of a user of the devices and systems described herein, with the term “proximal” being closer to the user's hand, and the term “distal” being further from the user's hand such as to often be located further within a body of the patient during use. The term “passive” is defined in reference to flow regulation mechanisms described herein, with the term “passive” meaning that the flow regulation mechanism does not employ a source of electromotive force.

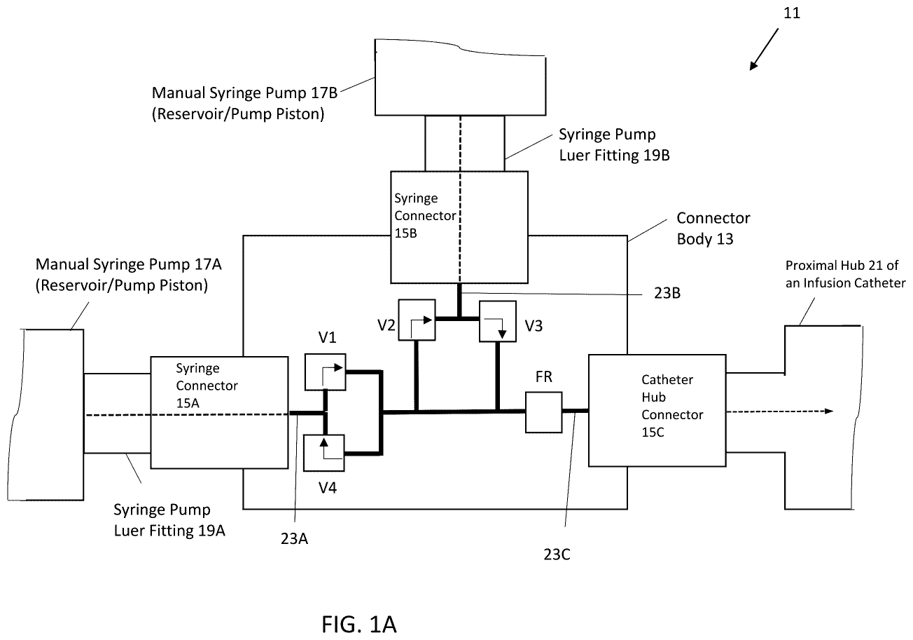

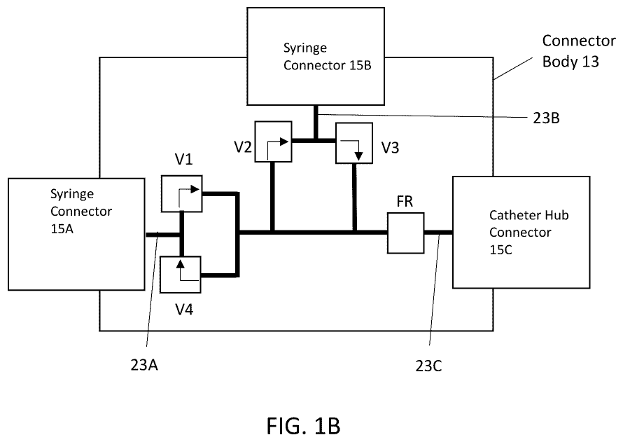

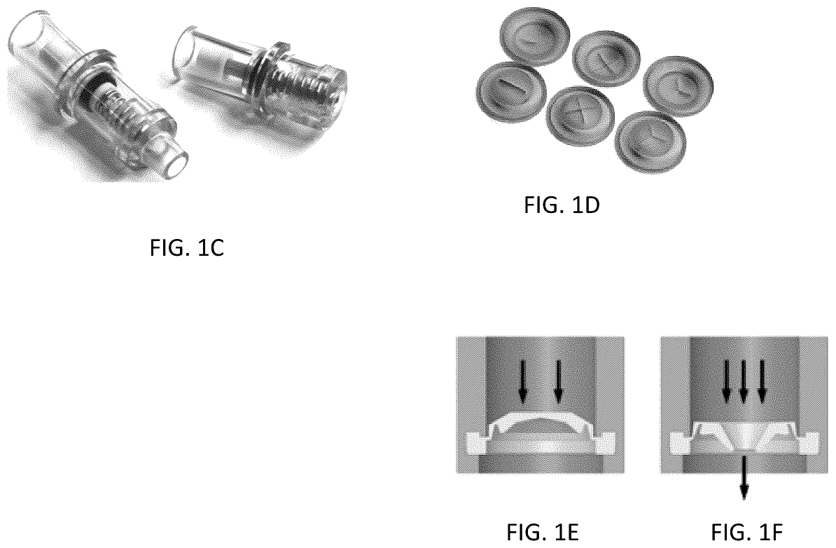

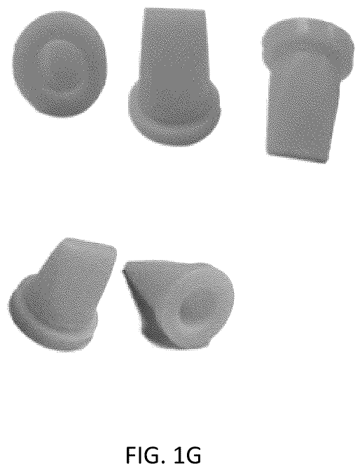

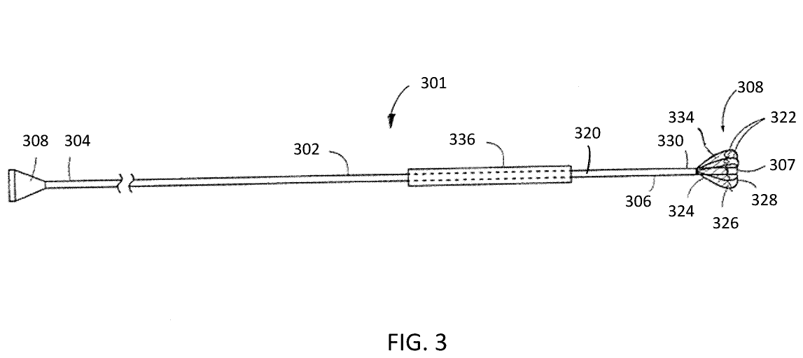

Turning to FIGS. 1A and 1B, a medical system 11 is provided that includes a connector body 13 with a first syringe connector 15A, a second syringe connector 15B and a catheter hub connector 15C integral to the connector body 13. The first syringe connector 15A can be configured to fluidly couple and detachably connect to a first manual syringe pump 17A, for example, using a luer fitting 19A at the distal tip of the first manual syringe pump 17A as shown. The second syringe connector 15B can be configured to fluidly couple and detachably connect to a second manual syringe pump 17B, for example, using a luer fitting 19B at the distal tip of the second manual syringe pump 17B as shown. The first and second manual syringe pumps 17A, 17B can be conventional manual syringe pumps each including a piston with a proximal handle that sealably slides axially within an annular pump body. The pump body defines a variable-size reservoir that holds fluid therein for outflow of fluid from an open distal tip as the piston moves distally toward the open distal tip, or inflow of fluid into the reservoir from the open distal tip as the piston moves proximally away from the open distal tip. The catheter hub connector 15C is configured to fluidly couple and detachably connect to the hub 21 of an infusion catheter as shown. The infusion catheter can be configured to deliver a therapeutic agent and/or a secondary fluid (such as a contrast agent for vascular imaging and visualization, a bolus of saline or other non-therapeutically active agent, or a different therapeutic agent) into the vascular system of a patient for treating disease of a target organ. For example, the hub 21 can correspond to the hub 308 of the exemplary infusion catheter of FIG. 3. Alternatively, other suitable infusion catheters can be used.

The first syringe connector 15A and the connector body 13 provide an internal flowpath (channel) 23A that branches and couples to two valves V1 and V4 that are integral to the connector body 13. Specifically, one branch of the flowpath (channel) 23A is fluidly coupled to the inlet of valve V1, while the other branch of the flowpath (channel) 23A is fluidly coupled to the outlet of valve V4. The valves V1 and V4 are also fluidly coupled to an internal flowpath (channel) 23C that extends through the connector body 31 and through the catheter hub connector 15C to the hub 21. Specifically, the outlet of valve V1 is fluidly coupled to the internal flowpath (channel) 23C, and the inlet of the valve V4 is fluidly coupled to the internal flowpath (channel) 23C.

Similarly, the second syringe connector 15B and the connector body 13 provide an internal flowpath (channel) 23B that branches and couples to two valves V2 and V3 that are integral to the connector body 13. Specifically, one branch of the flowpath (channel) 23B is fluidly coupled to the inlet of valve V3, while the other branch of the flowpath (channel) 23B is fluidly coupled to the outlet of valve V2. The valves V2 and V3 are also fluidly coupled to the internal flowpath (channel) 23C that extends through the connector body 31 and through the catheter hub connector 15C to the hub 21. Specifically, the outlet of valve V3 is fluidly coupled to the internal flowpath (channel) 23C, and the inlet of the valve V2 is fluidly coupled to the internal flowpath (channel) 23C.

FIG. 1B shows the components integral to the connector body 13 alone (without being connected to the two manual syringe pumps and the catheter hub). In embodiments, the connector body 13 can be hand-holdable and/or include one or more housing parts that enclose and/or mechanically support the first syringe connector 15A, the second syringe connector 15B, and the catheter hub connector 15C together with the flowpaths (channels) 23A, 23B, 23C and the valves V1, V2, V3 and V4 as shown. The flowpath(s)/channels 23A, 23B, 23C can be embodied by tubing and associated fluid couplers or other suitable fluid transfer structures and devices. The valves V1, V2, V3 and V4 can be embodied by medical-grade pressure-relief valves that are adapted to control the flow of fluid therethrough without reacting with and contaminating the fluid.

For example, the valves V1, V2, V3 and V4 can be a coil spring valve as shown in FIG. 1C. The coil spring valve employs a coil spring to provide a spring bias that maintains the valve in a closed state to block flow through the valve. The valve opens to permit flow through the valve when sufficient pressure at the inlet of the valve overcomes the spring bias provided by the coil spring. Alternatively the valve can employ a flat spring or other spring to provide the spring bias spring bias that maintains the valve in a closed state.

In another example, the valves V1, V2, V3 and V4 can be slit valves as shown in FIGS. 1D-1F. The slit valve employs an elastic diaphragm or sealing gasket with slits that are configured in a closed state to block flow through the valve as shown in FIG. 1E. The valve opens to permit flow through the valve when sufficient pressure at the inlet of the valve opens the slits of the diaphragm or gasket as shown in FIG. 1F.

In another example, the valves V1, V2, V3 and V4 can be elastomeric duck bill valves as shown in FIG. 1G. The elastomeric duck bill valve employs an elastomeric taper body that leads to an outlet that is configured in a closed state to block flow through the valve. The valve opens to permit flow through the valve when sufficient pressure at the inlet of the valve deforms the elastomeric tapered body and opens the outlet of the valve.

The system 11 can be configured to maintain consistency of delivery of a therapeutic agent and/or a secondary fluid into the vascular system of a patient in conjunction with manual pump action provided by user (physician) operation of one or both of the first and second manual syringe pumps 17A, 17B. In this manner, the system 11 can significantly reduce the complexity of the procedure while maintaining favorable distribution and uptake patterns observed for a desired infusion rate (or desired infusion rate window).

In embodiments, the valve V1 can be designed and configured to open at a predefined supply pressure SP_{V1} that produces the desired infusion rate. In this manner, when the pressure within the flowpath (channel) 23A coupled to the inlet of valve V1 is less than such predefined supply pressure SP_{V1}, the valve V1 will remain closed and block flow through the valve V1 and into the internal flowpath (channel) 23C that extends through the connector body 31 and through the catheter hub connector 15C to the hub 21. When the pressure within the flowpath (channel) 23A coupled to the inlet of valve V1 reaches or exceeds such predefined supply pressure SP_{V1}, the valve V1 will open and enable flow through the valve V1 and into and through the internal flowpath (channel) 23C that extends through the connector body 31 and through the catheter hub connector 15C to the hub 21.

In embodiments, the valve V2 can be designed and configured to open at a set supply pressure SP_{V2} above the predefined supply pressure SP_{V1} for valve V1 to bleed excess pressure/flow and permit flow into the reservoir of the second manual syringe pump 17B. In this manner, when the pressure within the flowpath (channel) 23C coupled to the inlet of valve V2 is less than such set supply pressure SP_{V2}, the valve V2 will remain closed and block flow through the valve V2 and into the internal flowpath (channel) 23B that extends through the connector body 31 and through the second syringe connector 15B to the second manual syringe pump 17B. When the pressure within the flowpath (channel) 23C coupled to the inlet of valve V2 reaches or exceeds such predefined supply pressure SP_{V2}, the valve V2 will open and enable flow through the valve V2 and into the internal flowpath (channel) 23B that extends through the connector body 31 and through the second syringe connector 15B to the second manual syringe pump 17B.

In embodiments, the valve V3 can be designed and configured to open at a predefined supply pressure SP_{V3} that produces the desired infusion rate. In this manner, when the pressure within the flowpath (channel) 23B coupled to the inlet of valve V3 is less than such predefined supply pressure SP_{V3}, the valve V3 will remain closed and block flow through the valve V3 and into the internal flowpath (channel) 23C that extends through the connector body 31 and through the catheter hub connector 15C to the hub 21. When the pressure within the flowpath (channel) 23B coupled to the inlet of valve V3 reaches or exceeds such predefined supply pressure SP_{V3}, the valve V3 will open and enable flow through the valve V3 and into the internal flowpath (channel) 23C that extends through the connector body 31 and through the catheter hub connector 15C to the hub 21.

In embodiments, the valve V4 can be designed and configured to open at a set supply pressure SP_{V4} above the predefined supply pressure SP_{V3} for valve V3 to bleed the excess pressure/flow and permit flow into the reservoir of the first manual syringe pump 17A. In this manner, when the pressure within the flowpath (channel) 23C coupled to the inlet of valve V4 is less than such set supply pressure SP_{V4}, the valve V4 will remain closed and block flow through the valve V4 and into the internal flowpath (channel) 23A that extends through the connector body 31 and through the first syringe connector 15A to the first manual syringe pump 17A. When the pressure within the flowpath (channel) 23C coupled to the inlet of valve V4 reaches or exceeds such predefined supply pressure SP_{V4}, the valve V4 will open and enable flow through the valve V4 and into the internal flowpath (channel) 23A that extends through the connector body 31 and through the first syringe connector 15A to the first manual syringe pump 17A.

In the configuration of FIGS. 1A and 1B, the valves V1, V2, V3, and V4 of the connector body 14 provide a passive pressure-controlled mechanical mechanism for regulating the flow of the therapeutic agent and/or secondary fluid supplied to the infusion catheter and delivered by the infusion catheter into the vascular system of the patient at or near a desired rate of infusion.

In embodiments, the connector body 13 of FIGS. 1A and 1B can optionally include a passive flow restrictor FR coupled to the flowpath downstream of the valves V1, V2, V3, and V4. The passive flow restrictor FR is a device that regulates the flow rate of fluid flow through the flowpath. The passive flow restrictor FR can employ a fixed-size orifice or plug or other suitable passive flow regulating mechanism. The passive flow restrictor can be configured such that slow flow rates of fluid through the connector body generate enough pressure to activate the first and second pressure relief valves V1 and V2.

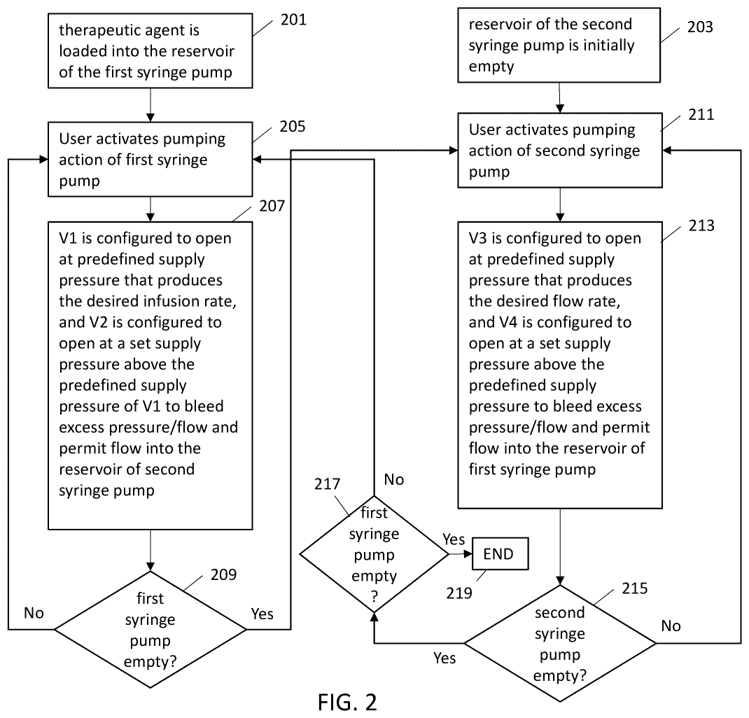

FIG. 2 illustrates the operation of the system 11 of FIG. 1 during a medical procedure that delivers a therapeutic agent into the vascular system of a patient. As part of such medical procedure, the infusion catheter is introduced into a target vessel of the vascular system by a user (physician). In embodiments, the target vessel can extend into or near a tumor or other diseased tissue. The target vessel may feed or drain from any of various organs, including, but not limited to, the pancreas, spleen, gastrointestinal tract, liver, lung, uterus, prostate, or brain, as well as target vessels communicating with head and neck tumors. The target vessel may also be in communication with other organs or tissues of interest for treatment in other parts of the body. In embodiments, the treatment system may be introduced into or adjacent the target vessel non-endovascularly.

In block 201, the therapeutic agent is loaded into the reservoir of the first manual syringe pump 17A.

In block 203, the reservoir of the second manual syringe pump 17B is initially empty.

In block 205, the user (physician) manually activates pumping action of the first syringe pump 17A.

In blocks 207, during the manual pumping action of block 205, the valve V1 is configured to open at predefined supply pressure that produces the desired infusion rate, and the valve V2 is configured to open at a set supply pressure above the predefined supply pressure of V1 to bleed any excess pressure/flow and permit excess flow of the therapeutic agent into the reservoir of second syringe pump 17B. In this configuration, the manual pumping action of the first syringe pump 17A delivers flow of the therapeutic agent through valve V1 into and through the flowpath (channel) 23C and into and through the catheter hub connector 15C for delivery by the infusion catheter at or near the desired rate of infusion. Any excess pressure and flow of therapeutic agent into the flowpath (channel) 23C that is caused by the manual pumping action of the first syringe pump 17A will be diverted to the reservoir of the second syringe pump 17B via valve V2.

In block 209, the pumping action of the user and delivery of therapeutic agent from the reservoir of the first syringe pump of blocks 205 and 207 are continued until the reservoir of the first syringe pump is empty (or near empty) and the operations continue to block 211 for the case where the reservoir of the second syringe pump is not empty (or near empty).

In block 211, the user (physician) manually activates pumping action of the second syringe pump 17B.

In blocks 213, during the manual pumping action of block 211, the valve V3 is configured to open at predefined supply pressure that produces the desired infusion rate, and the valve V4 is configured to open at a set supply pressure above the predefined supply pressure of V3 to bleed any excess pressure/flow and permit excess flow of the therapeutic agent into the reservoir of first syringe pump 17A. In this configuration, the manual pumping action of the second syringe pump 17B delivers flow of the therapeutic agent through valve V3 into and through the flowpath (channel) 23C and into and through the catheter hub connector 15C for delivery by the infusion catheter at or near the desired rate of infusion. Any excess pressure and flow of therapeutic agent into the flowpath (channel) 23C that is caused by the manual pumping action of the second syringe pump 17B will be diverted to the reservoir of the first syringe pump 17A via valve V4.

In block 215, the pumping action of the user and delivery of therapeutic agent from the reservoir of the second syringe pump of blocks 211 and 213 are continued until the reservoir of the second syringe pump is empty (or near empty) and the operations can revert back to block 205 for the case where the reservoir of the first syringe pump is not empty (or near empty) at block 217. The process ends once both syringes have been emptied of the prescribed delivery dose at block 219 (which could be set to leave a predetermined volume of therapeutic agent in the first or second syringe).

In this manner, the procedure can maintain consistency of delivery of the therapeutic agent into the vascular system of a patient at or near the desired rate of infusion in conjunction with manual pump action provided by user (physician) operation of one or both of the first and second manual syringe pumps 17A, 17B (including alternating use of the first and second manual syringe pumps 17A, 17B) until the infusion is completed. Furthermore, the therapeutic agent can include suspended particles (such as macroaggregated albumen, resin spheres, glass spheres, gel beads, other embolic particles) and/or an emulsion of liquids that normally do not mix (typically in lipiodol), In this case, the regulated flow provided by the connector body 13 can keep the suspended particles and/or emulsified liquids evenly dispersed in the flow through the connector body during administration.

In embodiments, the desired infusion rate can range from 0.1 milliliters/second to 4 milliliters per second or more, with the supply pressure at the catheter hub ranging from 9 psi to 2000 psi for such infusion rates. These supply pressure values can be used to design the valves and flowpaths of the connector body for a desired infusion rate, and also select the suitable manual syringe pump. Specifically, the maximum pressure of a manual syringe pump can vary by size of the syringe pump, and thus the size of the manual syringe pump should be selected to have a maximum pressure that matches or exceeds the supply pressure that provides the desired rate of infusion. For example, if the desired rate of infusion is 0.5 milliliters per second at a supply pressure between 90 to 99 psi, then manual syringe pumps with a maximum pressure that exceeds 100 psi (e.g., conventional syringe pumps that are 10 cc or smaller) can be used in the procedure.

In the procedure of FIG. 2, a high pressure flow of a secondary fluid (such as a contrast agent for vascular imaging and visualization, a bolus of saline or other non-therapeutically active agent, or a different therapeutic agent) can also be delivered through the infusion catheter of the system 11 into the vascular system of the patient. For example, the secondary fluid can be loaded into the reservoir of an additional manual syringe pump (not shown). The additional manual syringe pump can be a conventional manual syringe pump as described herein. The additional manual syringe pump can be fluidly coupled and detachably connected to one of the first and second syringe connectors (for example, 15A), with the other of the first and second syringe connectors (for example, 15B) is capped to block flow through the other syringe connector. The user (physician) can manually activate pumping action of the additional manual syringe pump to deliver the secondary fluid from additional manual syringe pump into and through the flowpath (channel) 23C and into and through the catheter hub connector 15C for delivery by the infusion catheter into the vascular system of the patient. In this configuration, the relief valve (V2 or V4) whose outlet leads to the capped syringe connector does not operate to bleed excess pressure/flow. Thus, the manual pumping action of the additional manual syringe pump can be used to deliver a flow of the secondary fluid into and through the infusion catheter into the vascular system of the patient at a pressure at or above the desired rate of infusion of the therapeutic agent. In embodiments, the manual pumping action of the additional manual syringe pump can be used to deliver a flow of the secondary fluid into and through the infusion catheter into the vascular system of the patient after infusion of the therapeutic agent, and such secondary fluid delivery can be performed at a high pressure/flow above the desired rate of infusion of the therapeutic agent such that the flow of the secondary fluid pushes the therapeutic agent in the vascular system toward diseased tissue.

An embodiment of an exemplary infusion catheter 101 is shown in FIG. 3, which includes a flexible tubular body 302 having a proximal end 304 and a distal end 306. The tubular body 302 preferably has a length between two and eight feet long, and preferably has an outer diameter of between 0.67 mm and 3 mm (corresponding to catheter sizes 2 French to 12 French). The tubular body 302 preferably includes an inner liner, an inner braid, and an outer coating. By way of example, the liner may be made of fluorinated polymer such as polytetrafluoroethylene (PTFE) or fluorinated ethylene propylene (FEP). By way of example, the braid is made of metal such as stainless steel or nickel titanium alloy, or a polymer such as polyethylene terephthalate (PET) or liquid crystal polymer. By way of example, the outer coating is made of a polyether block amide thermoplastic elastomeric resin such as Pebax®, polyurethane, polyamide, copolymers of polyamide, polyester, copolymers of polyester, fluorinated polymers, such as PTFE, FEP, polyimides, polycarbonate, or any other suitable material, or any other standard or specialty material used in making catheters used in the bloodstream.

The proximal end 304 of the tubular body 302 is preferably provided with a hub 308. An infusion lumen 320 extends internally through the hub 308 and through the interior of the tubular body 302 to the distal end 306 and exits at an open distal tip or orifice 307. The hub 308 and infusion lumen 320 are adapted for delivery of the therapeutic agent from outside the body of a patient into the vascular system (artery or vein) of the patient for treating disease of a target organ. The hub 308 can also be adapted to facilitate advancement of a guidewire through the infusion lumen 320. Any hub 308 suitable for at least facilitating delivery of a therapeutic into the infusion lumen can be utilized.

An occluder 308 is preferably coupled to the distal end 306 of the tubular body 302. The occluder prevents reflux of the therapeutic agent between the vessel wall and the catheter to non-treatment tissues during delivery of the agent. Occluders can be either static or dynamic during use. Static occluders include inflatable balloons. Dynamic occluders include microvalves which expand and contract within the vessel in relation to the surrounding fluid pressure variations. When the therapeutic agent is infused, the microvalve opens and contacts the vascular wall to block the reflux of the therapeutic agent that would otherwise be caused by vascular resistance. In a preferred embodiment, the occluder 308 includes elastic strands 322 that each include a proximal portion 324, a central portion 326, and a distal portion 328. The proximal portions 324 are attached circumferentially about at an outer surface 330 of the tubular body 302 at a location proximal of the open distal tip or orifice 307. The central portions 326 extend radially outward and toward the open distal tip or orifice 307. The distal portions 328 are inverted back into the occluder 308 and coupled circumferentially about the outer surface 330. The proximal and central portions 324, 326 are coated with a polymeric filter coating 334 that extends between and across the strands 322. The distal portions 328 of the strands 322 are uncoated. This infusion catheter 301 is commercially produced and distributed as the TRINAV® infusion system by Trisalus Life Sciences, Inc., Westminster, CO. In use, the infusion catheter 301 can be deployed from an introducer sleeve (shown schematically as 336) at a target vessel location; but is not required to be used with an introducer sleeve. Rather, the infusion system can be advanced over a guidewire without any introducer sleeve, providing good results with trackability. Upon introduction in the vessel, the valve occluder 308 has been shown to dynamically operate in sync with the cardiac cycle of the patient and preserve more than seventy percent of antegrade blood flow in a vessel past the microvalve occluder, while providing intended retrograde blockade of therapeutics. Further, the design permits atraumatically increasing the pressure of therapeutic infusion into local resistive tumor vessels to enable deeper perfusive delivery of therapeutics. In alternate embodiments, one or more other infusion catheters can be used as part of the systems and methods described herein.

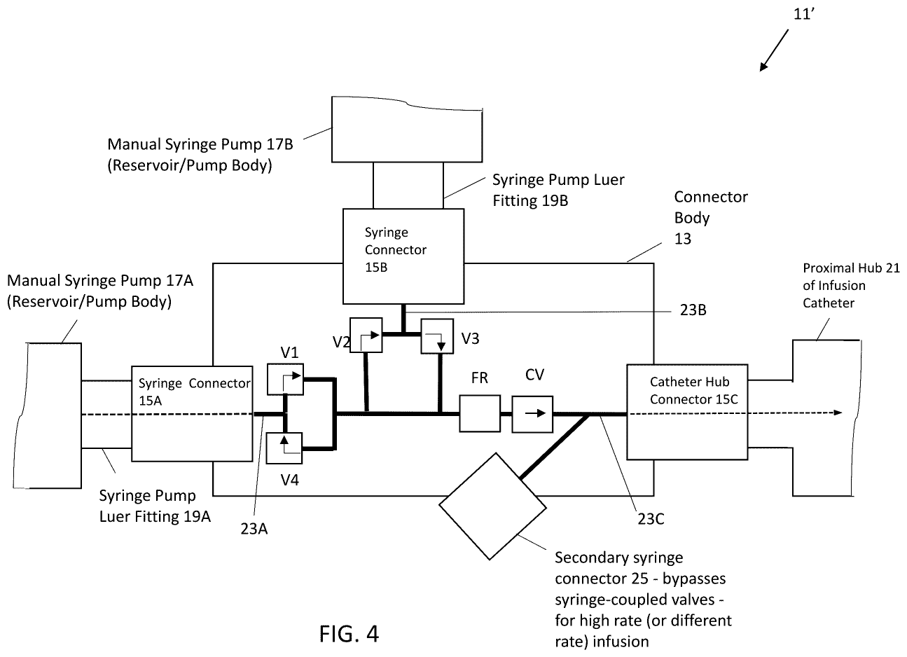

FIG. 4 illustrates another embodiment of a medical system 11′ that is similar to the embodiment describes above with respect to FIGS. 1A and 1B with the addition of a secondary syringe connector 25 and check valve CV integral to the connector body 13. The secondary syringe connector 25 can be capped to block flow through the secondary connector 25 during pumping operation of the first and/or second manual syringe pumps 17A, 17B. Alternatively, the secondary syringe connector 25 can be fluidly coupled and detachably connected to an additional manual syringe pump (not shown) whose reservoir is loaded with a secondary fluid (such as a contrast agent for vascular imaging and visualization, a bolus of saline or other non-therapeutically active agent, or a different therapeutic agent). The secondary syringe connector 25 and the connector body 13 provide an internal flowpath that is fluidly coupled to the internal flowpath (channel) 23C that extends through the connector body 13 and through the catheter hub connector 15C to the hub 21 as shown. The check valve CV is fluidly coupled to the flowpath (channel) 23C upstream from the flowpath leading to the secondary connector 25 and downstream from the flowpaths leading to the valves V1, V2, V3, and V4 as shown. In this configuration, the check valve CV permits flow in one direction (i.e., flow toward the catheter hub connector 15C) and blocks flow in the opposite direction and thus blocks the flow of the secondary fluid back toward the valves V1, V2, V3, V4 and the first and second manual syringe pumps 17A, 17B. The optional passive flow restrictor (FR) is coupled to the flowpath downstream of the valves V1, V2, V3, and V4. The passive flow restrictor FR is a device that regulates the flow rate of fluid flow through the flowpath. The passive flow restrictor FR can employ a fixed-size orifice or plug or other suitable passive flow regulating mechanism. The passive flow restrictor FR can be configured such that slow flow rates of fluid through the connector body 13 generate enough pressure to activate the first and second pressure relief valves V1, V2. In other embodiments, the check valve CV and passive flow restrictor FR can be provided by a unitary device that is configured as both a check valve and passive flow restrictor as described herein.

The system 11′ can be used to perform the procedure of FIG. 2 as described herein. Additionally or alternatively, manual pump activation of the additional manual syringe pump fluidly coupled to the secondary syringe connector 25 can supply a flow of the secondary fluid into and through the flowpath (channel) 23C and into and through the catheter hub connector 15C for delivery by the infusion catheter into the vascular system of the patient. In this configuration, the check valve CV blocks the flow of the secondary fluid upstream toward the valves V1, V2, V3 and V4 such that the relief valves V2, V4 (whose outlets leads to the syringe connectors 15A, 15B) do not operate to bleed excess pressure/flow. The manual pumping action of the additional syringe pump can be used to deliver a flow of the secondary fluid into and through the infusion catheter into the vascular system of the patient at a pressure at or above the desired rate of infusion of the therapeutic agent. In embodiments, the manual pumping action of the additional syringe pump can be used to deliver a flow of the secondary fluid into and through the infusion catheter into the vascular system of the patient after infusion of the therapeutic agent, and such secondary fluid delivery can be performed at a high pressure/flow above the desired rate of infusion of the therapeutic agent such that the flow of the secondary fluid (e.g., a bolus of saline) pushes the therapeutic agent in the vascular system toward diseased tissue. In other embodiments, the manual pumping action of the additional syringe pump can be used to deliver the secondary fluid at a different rate of infusion, which can be below or above the desired rate of infusion of the therapeutic agent.

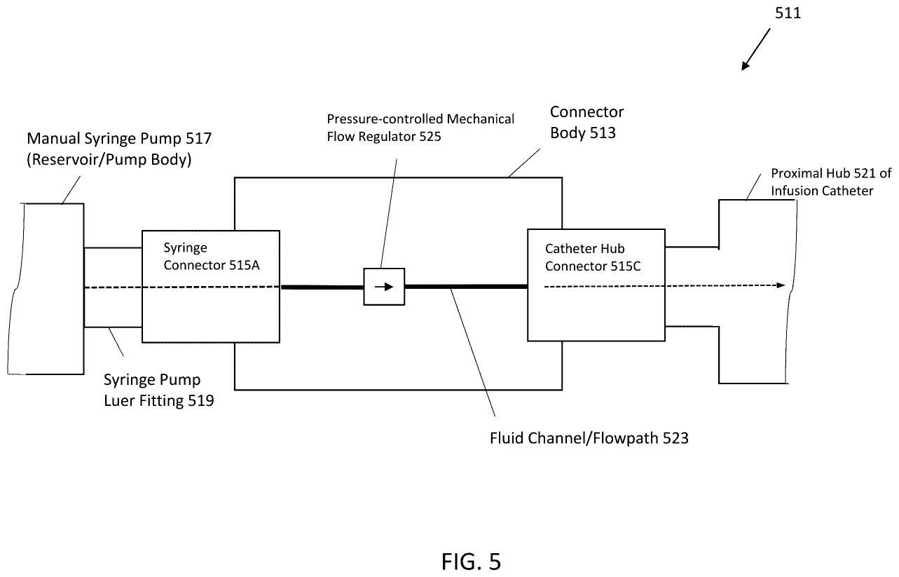

Turning to FIG. 5, a medical system 511 is provided that includes a connector body 513 with a syringe connector 515A and a catheter hub connector 515C integral to the connector body 513. The syringe connector 515A can be configured to fluidly couple and detachably connect to a manual syringe pump 517, for example, using a luer fitting 519 at the distal tip of the manual syringe pump 517 as shown. The manual syringe pump 517 can be a conventional manual syringe pump as described herein. The catheter hub connector 515C can be configured to fluidly couple and detachably connect to the hub 521 of an infusion catheter as shown. The infusion catheter can be configured to deliver a therapeutic agent and/or a secondary fluid (such as a contrast agent for vascular imaging and visualization, a bolus of saline or other non-therapeutically active agent, or a different therapeutic agent) into the vascular system of a patient for treating disease of a target organ. For example, the hub 521 can correspond to the hub 308 of the exemplary infusion catheter of FIG. 3. Alternatively, other infusion catheters can be used.

The syringe connector 515A and the connector body 513 provide an internal flowpath (channel) 523 that extends through the connector body 513 and through the catheter hub connector 515C to the hub 521. The internal flowpath (channel) 523 includes a passive pressure-controlled mechanical flow regulator 525 that is configured to regulate the flow of the therapeutic agent supplied to the infusion catheter and delivered by the infusion catheter into the vascular system of the patient at or near a desired rate of infusion.

In embodiments, the connector body 513 can be hand-holdable and/or include one or more housing parts that enclose and/or mechanically support the syringe connector 515A and the catheter hub connector 515C together with the flowpath (channel) 523 and the regulator 525. The flowpath/channel 523 can be embodied by tubing and associated fluid couplers or other suitable fluid transfer structures and devices.

The system 511 can be configured to maintain consistency of delivery of a therapeutic agent and/or a secondary fluid (such as a contrast agent for vascular imaging and visualization, a bolus of saline or other non-therapeutically active agent, or a different therapeutic agent) into the vascular system of a patient in conjunction with manual pump action provided by user (physician) operation of one or more manual syringe pumps. In this manner, the system 511 can significantly reduce the complexity of the procedure while maintaining favorable distribution and uptake patterns observed for a desired infusion rate (or desired infusion rate window). The therapeutic agent can include suspended particles (such as macroaggregated albumen, resin spheres, glass spheres, gel beads, other embolic particles) and/or an emulsion of liquids that normally do not mix (typically in lipiodol). Furthermore, the regulated flow provided by the system 511 can be calibrated to a flow rate range based on the size of the syringe and resistance of the catheter selected.

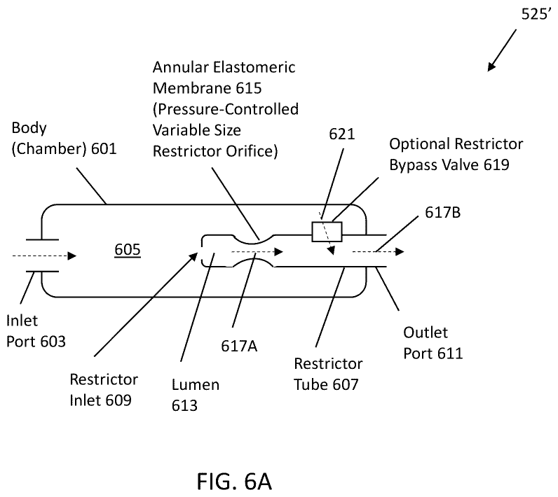

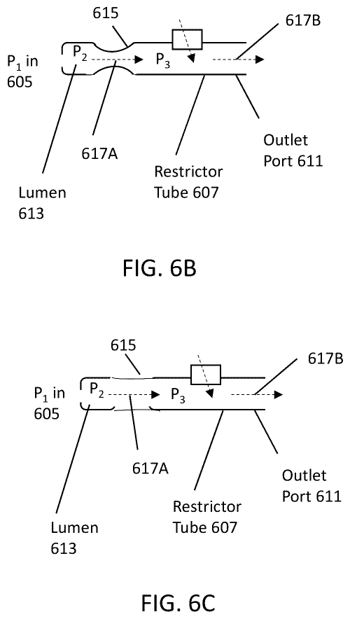

FIG. 6A illustrates an exemplary embodiment of a passive pressure-controlled mechanical flow regulator 525′ suitable for use as the regulator 525 in the system 511 of FIG. 5. The regulator 525′ includes a hollow body (or chamber) 601 with an inlet port 603 leading into the interior space 605 of the hollow body 601. The body 601 further supports or defines a restrictor tube 607 that extends into the interior space 605 of the chamber. One end of the restrictor tube 607 defines a restrictor inlet 609 that is located within the interior space 605. The opposite end of the restrictor tube 607 defines an outlet port 611 that extends from the body 601. The restrictor tube 607 defines a lumen 613 that extends from the restrictor inlet 609 to the outlet port 611 with a portion of the lumen 613 defined by an annular elastomeric (flexible) membrane 615 that is configured as a pressure-controlled variable size restrictor orifice. The inlet port 603 and the outlet port 611 can be fluidly coupled to the internal flowpath (channel) 523 that extends through the connector body 531 of FIG. 5. In this configuration, the therapeutic agent supplied to the flowpath (channel) 523 fills the interior space 605 of the body 601. The restrictor inlet 609 is configured to provide a pressure drop between the interior space 605 and the lumen 613. This pressure drop causes the annular elastomeric membrane 615 to resiliently deform or deflect inward toward the central axis of the lumen 613 and function as a restrictor orifice for the flow of therapeutic agent through the lumen 613 and exit from the outlet port 613 as illustrated by arrows 617A and 617B. In embodiments, the resilient deformation or deflection of the elastomeric membrane 615 can occur in a radially inward direction, but such deformation or deflection is not limited to this configuration. For example, the elastomeric membrane 615 can have a linear design that pinches down between two surfaces or pinches down on a hard surface. The deformation or deflection of the annular elastomeric membrane 615 and thus the size of the restrictor orifice can vary based on pumping pressure. The restrictor inlet can be sized smaller than the lumen of the infusion catheter and configured to provide an initial flow restriction at relatively low pressures. The annular elastomeric membrane 615 provides a secondary flow restriction at higher pressure.

FIG. 6B illustrates a configuration of the annular elastomeric membrane 615 at an initial high pressure state where the pressure P1 in the interior space 605 is greater than or equal to the pressure P2 in the lumen 613 upstream of the annular elastomeric membrane 615, which is greater than the pressure P3 in the lumen 613 downstream of the annular elastomeric membrane 615. In this configuration, P1>P2>P3 and the annular elastomeric membrane 615 experiences deformation proportional to the magnitude of the difference in pressure between P1 P2 relative to P3. In this manner, the annular elastomeric membrane 615 of the restrictor tube 607 regulates the flow of the therapeutic agent supplied to the infusion catheter and delivered by the infusion catheter into the vascular system of the patient at or near a desired rate of infusion.

FIG. 6C illustrates a configuration of the annular elastomeric membrane 615 at a low pressure steady state where the pressure P1 in the interior space 605 is nearly equal to the pressure P2 in the lumen 613 upstream of the annular elastomeric membrane 615, which is nearly equal to the pressure P3 in the lumen 613 downstream of the annular elastomeric membrane 615. In this configuration, P1 is nearly equal to P2 which is nearly equal to P3 and the annular elastomeric membrane 615 experiences minimal deformation or deflection. The flow of the therapeutic agent is then governed by the restrictor inlet 609 and the resistance of the infusion system.

The restrictor tube 607 can also include an optional bypass valve 619 disposed downstream from the annular elastomeric membrane 615. The bypass valve 619 can be designed and configured to open at a predefined supply pressure within the interior space 605 that exceeds the operating pressure for desired flow through the restrictor orifice provided by membrane 615 to the outlet port 611. In this manner, when the pressure within the interior space 605 is less than such predefined supply pressure, the bypass valve 619 will remain closed and block flow through the bypass valve 619 and into the lumen 613 that extends through the restrictor tube 607 to the outlet port 611. When the pressure within the interior space 605 reaches or exceeds such predefined supply pressure, the bypass valve 619 will open and enable flow through the bypass valve 619 and into the lumen 613 that extends through the restrictor tube 607 to the outlet port 611 as depicted by arrows 621 and 617B. The bypass valve 619 can be embodied by a pressure-relief valve that are adapted to control the flow of fluid therethrough without reacting with and contaminating such fluid as described herein.

In embodiments, the connector 515A of the system of FIGS. 5 and 6 can be coupled to a first manual syringe pump 517 whose reservoir is loaded with a therapeutic agent. The first manual syringe pump 517 can be a conventional manual syringe pump as described herein. The catheter hub connector 515C can be configured to fluidly couple and detachably connect to the hub 521 of an infusion catheter as shown. For example, the hub 521 can correspond to the hub 308 of the exemplary infusion catheter of FIG. 3. The syringe connector 515A and the connector body 513 provide an internal flowpath that is fluidly coupled to the internal flowpath (channel) 523 that extends through the connector body 513 and through the catheter hub connector 515C to the proximal hub 521 of the infusion catheter as shown. The first manual syringe pump 517 is manually actuated to pump the therapeutic agent into and through the internal flowpath (channel) 523. The regulator 525′ of FIG. 6 regulates the flow of the therapeutic agent supplied to the infusion catheter and delivered by the infusion catheter into the vascular system of the patient at or near a desired rate of infusion.

Additionally, or alternatively, the connector 515A of the system of FIGS. 5 and 6 can be coupled to an additional manual syringe pump (e.g., a second manual syringe pump similar to 517) whose reservoir is loaded with a secondary fluid (such as a contrast agent for vascular imaging and visualization, a bolus of saline or other non-therapeutically active agent, or a different therapeutic agent). The additional manual syringe pump can be a conventional manual syringe pump as described herein. The catheter hub connector 515C can be configured to fluidly couple and detachably connect to the hub 521 of the infusion catheter as shown. In this configuration, the syringe connector 515A and the connector body 513 provide an internal flowpath that is fluidly coupled to the internal flowpath (channel) 523 that extends through the connector body 513 and through the catheter hub connector 515C to the proximal hub 521 of the infusion catheter as shown. The additional syringe pump is manually actuated to pump the secondary fluid into and through the internal flowpath (channel) 523. The bypass valve 619 of the restrictor tube 607 can be configured to open and enable flow through the bypass valve 619 and into the lumen 613 that extends through the restrictor tube 607 to the outlet port 611 at the predefined pressure and infusion rate desired for the infusion of the secondary fluid. In this configuration, the manual pumping action of the additional syringe pump can be used to deliver a flow of the secondary fluid into and through the infusion catheter into the vascular system of the patient at a pressure at or above the desired rate of infusion of the therapeutic agent. In embodiments, the manual pumping action of the additional syringe pump can be used to deliver a flow of the secondary fluid into and through the infusion catheter into the vascular system of the patient after infusion of the therapeutic agent, and such secondary fluid delivery can be performed at a high pressure/flow above the desired rate of infusion of the therapeutic agent such that the flow of the secondary fluid (e.g., bolus of saline) pushes the therapeutic agent in the vascular system toward diseased tissue. In other embodiments, the manual pumping action of the additional syringe pump can be used to deliver the secondary fluid at a different rate of infusion, which can be below or above the desired rate of infusion of the therapeutic agent.

As noted above, the maximum pressure of a manual syringe pump varies by size of the syringe pump, and thus the size of the manual syringe pumps can be selected to have a maximum pressure that matches the supply pressure that provides the desired rate of infusion for the therapeutic agent and the secondary fluid, respectively. For example, if the desired rate of infusion of therapeutic agent is 0.5 milliliters per second at a supply pressure between 90 to 99 psi and the desired rate of infusion of the secondary fluid is 1 milliliter per second at a supply pressure of 155-180 psi, then a manual syringe pump with a maximum pressure at or above 100 psi but below the 155-185 psi range (e.g., conventional 10 cc syringe pump) can be used for infusion of the therapeutic agent, and a manual syringe pump with a maximum pressure above the 155-185 psi range (e.g., conventional 6 cc syringe pump) can be used for infusion of the secondary fluid. In this example, the bypass value 619 can be configured to open and provide flow through the bypass valve 619 at pressures in the supply pressure range of 155-180 psi for infusion of the secondary fluid.

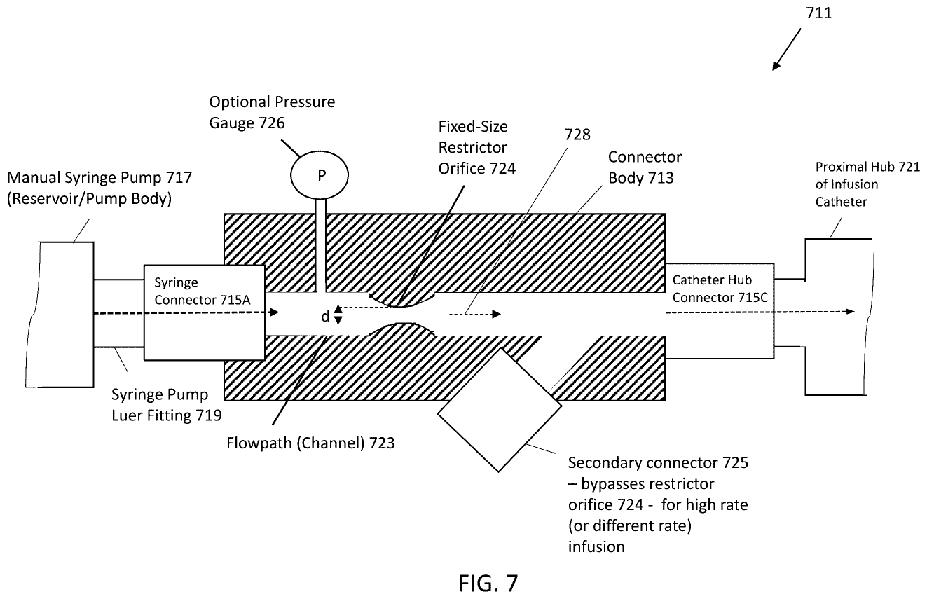

Turning to FIG. 7, a medical system 711 is provided that includes a connector body 713 with a syringe connector 715A and a catheter hub connector 715C integral to the connector body 713. The syringe connector 715A can be configured to fluidly couple and detachably connect to a manual syringe pump 717, for example, using a luer fitting 719 at the distal tip of the manual syringe pump 717 as shown. The catheter hub connector 715C can be configured to fluidly couple and detachably connect to the hub 721 of an infusion catheter as shown. The infusion catheter can be configured to deliver a therapeutic agent and/or a secondary fluid (such as a contrast agent for vascular imaging and visualization, a bolus of saline or other non-therapeutically active agent, or a different therapeutic agent) into the vascular system of a patient for treating disease of a target organ. For example, the hub 721 can correspond to the hub 308 of the exemplary infusion catheter of FIG. 3.

The syringe connector 715A and the connector body 713 provide an internal flowpath (channel) 723 that extends through the connector body 713 and through the catheter hub connector 715C to the hub 721. The internal flowpath (channel) 523 includes a fixed-sized restrictor orifice 724, which is configured as a passive pressure-controlled mechanical flow regulator that regulates the flow of the therapeutic agent or the secondary fluid to the infusion catheter and delivered by the infusion catheter into the vascular system of the patient at or near a desired rate of infusion. An optional pressure gauge can be provided to measure and display pressure of the fluid supplied to the flowpath (channel) 723. The display of such pressure measurements can be used to provide feedback to the user (physician) when actuating the manual syringe pump 717 such that the manual pumping action can be performed in a desired operating pressure range.

In embodiments, the connector body 713 can be hand-holdable and/or include one or more housing parts that enclose and/or mechanically support the syringe connector 715A and the catheter hub connector 715C together with the flowpath (channel) 723 and the fixed-sized restrictor orifice 724. The flowpath/channel 723 and the fixed-sized restrictor orifice 724 can be embodied by tubing and associated fluid couplers and nozzle structures or other suitable fluid transfer structures and devices.

The system 711 can be configured to maintain consistency of delivery of a therapeutic agent and/or a secondary fluid (such as a contrast agent for vascular imaging and visualization, a bolus of saline or other non-therapeutically active agent, or a different therapeutic agent) into the vascular system of a patient in conjunction with manual pump action provided by user (physician) operation of one or more manual syringe pumps. In this manner, the system 711 can significantly reduce the complexity of the procedure while maintaining favorable distribution and uptake patterns observed for a desired infusion rate (or desired infusion rate window). Furthermore, the therapeutic agent can include suspended particles (such as macroaggregated albumen, resin spheres, glass spheres, gel beads, other embolic particles) and/or an emulsion of liquids that normally do not mix (typically in lipiodol).

In embodiments, the fixed size d of the opening of the restrictor orifice 724 can be designed to regulate the flow of the therapeutic agent or the secondary fluid through the flowpath (channel) 723 at or near a desired rate of infusion, provided that fluid viscosity and catheter lumen diameter are known and the manual pump action of the syringe pump is performed in a predefined operating pressure range. This flow is depicted as arrow 728 and continues through the catheter hub connector 715C to the infusion catheter and delivered by the infusion catheter into the vascular system of the patient at or near the desired rate of infusion.

In embodiments, a kit of connector bodies 713 can be provided where each connector body of the kit is configured with a fixed size d of the opening of the restrictor orifice 724 that support desired rates of infusion for fluids with different fluid viscosities and/or with different catheter lumen diameters and/or with different manual syringe pumps with different operating pressure ranges. For example, a first connector body of the kit can be configured with a fixed size d1 of the opening of the restrictor orifice 724 that supports a desired rate of infusion for a first therapeutic agent of know viscosity (e.g., medium viscosity) with a first predefined catheter lumen diameter (e.g., 0.021 inches) with a 3 cc syringe pump operating in a pressure range around 200-250 psi. In another example, a second connector body of the kit can be configured with a fixed size d2 of the opening of the restrictor orifice 724 that supports a desired rate of infusion for a second therapeutic agent of know viscosity (e.g., high viscosity) with a second predefined catheter lumen diameter (e.g., 0.025 inches) with a 10 cc syringe pump operating in a pressure range around 100-125 psi. In yet another example, a third connector body of the kit can be configured with a fixed size d3 of the opening of the restrictor orifice 724 that supports a desired rate of infusion for saline fluid of know viscosity (e.g., low viscosity) with a third predefined catheter lumen diameter (e.g., 0.019 inches) with a 1 cc syringe pump operating in a pressure range around 500-750 psi. The different connector bodies of the kits can be labeled with visual markers or a predefined color scheme that can assist a user in selecting the connector body of the kit that matches the infusion system and procedure that will be used for treatment of a patient. The display of the pressure measurements provided by optional pressure gauge 726 can be used to provide feedback to the user (physician) when actuating the manual syringe pump 717 such that the manual pump action can be performed in a desired operating pressure range.

The selection of the connector body of the kit that matches the infusion system and procedure that will be used for treatment of a patient can also be aided by a lookup table. Table 1 provides an example connector body lookup table.

TABLE 1
Connector Body Lookup Table

| | | | | Connector |
|---|---|---|---|---|
| Dynamic Fluid | Infusion | | | Body (with |
| Viscosity | Catheter | Syringe | Operating | appropriate |
| (millipascal- | Lumen | Pump | Pressure | restrictor |
| second (mPa · s)) | Diameter | Size | Range | orifice size) |
| Therapeutic | 0.021 | 3cc | 200-250 | Label A, or |
| Agent A | inches | Syringe | psi | color A, with |
| (medium dynamic | | Pump | | restrictor |
| fluid viscosity. | | | | orifice size |
| e.g., lower | | | | of d1 |
| portion of range | | | | |
| 2.8-28.8 mPa · s) | | | | |
| Therapeutic | 0.025 | 10cc | 100-125 | Label B, or |
| Agent B | inches | Syringe | psi | color B, with |
| (high dynamic | | Pump | | restrictor |
| fluid viscosity. | | | | orifice size |
| e.g., upper | | | | of d2 |
| portion of range | | | | |
| 2.8-28.8 mPa · s) | | | | |
| Saline | 0.019 | 1cc | 500-700 | Label C, or |
| (low dynamic | inches | Syringe | psi | color C, with |
| fluid viscosity, | | Pump | | restrictor |
| e.g., 1.02 mPa · s) | | | | orifice size |
| | | | | of d3 |

The lookup table can be provided by a printed card that is packaged or distributed with the kit. Alternatively, the lookup table can be provided via a graphical user interface from a software application executing on a computing device. For example, the lookup table can be displayed as part of a graphical user interface provided by a web browser application or smartphone application.

There have been described and illustrated herein embodiments of systems and methods for therapeutic delivery, and in embodiment pressure-enabled therapeutic delivery. While particular embodiments of the invention have been described, it is not intended that the invention be limited thereto, as it is intended that the invention be as broad in scope as the art will allow and that the specification be read likewise. Thus, while the systems and methods are primarily adapted for therapeutic treatment of humans, it has been demonstrated on porcine tissues and organs, and can be used for the treatment of mammals, in general. Both humans and animals shall be considered ‘patients’ for purpose of this disclosure. Also, the therapy delivered herein can be a single therapeutic agent, or a combination of therapeutic agents. It will therefore be appreciated by those skilled in the art that, yet other modifications could be made to the provided invention without deviating from its scope as claimed.

What is claimed is:

1. A device for use with at least one infusion catheter for treatment of a patient, wherein the at least one infusion catheter has a proximal end with a hub, a distal end with a distal tip, and a lumen extending from the proximal end to the distal tip, the device comprising:
a connector body having a first connector for fluidly coupling and detachably connecting to the hub of the at least one infusion catheter, a second connector for fluidly coupling and detachably connecting to at least one manual syringe pump, and a passive pressure-controlled mechanical flow regulator that regulates fluid flow supplied by manual pump action of the least one manual syringe pump to the infusion catheter and delivered by the infusion catheter into the vascular system of the patient.

2. The device of claim 1, wherein:
the passive pressure-controlled mechanical flow regulator is configured to deliver a therapeutic agent or a secondary fluid supplied under pressure by pumping action of the least one manual syringe pump into and through the lumen of the infusion catheter at a desired rate of infusion.

3. The device of claim 1, wherein:
the connector body further includes a first flowpath that extends through the first connector and is fluidly coupled to the lumen of the infusion catheter during use and a second flowpath that extends through the second connector and is fluidly coupled to the least one manual syringe pump during use, wherein the passive pressure-controlled mechanical flow regulator is fluidly coupled to both the first flowpath and the second flowpath.

4. The device of claim 3, wherein:
the connector body further includes a third connector for fluidly coupling and detachably connecting to at least one additional manual syringe pump and a third flowpath that extends through the third connector and is fluidly coupled to the least one additional manual syringe pump during use, wherein the passive pressure-controlled mechanical flow regulator is fluidly coupled to the third flowpath.

5. The device of claim 4, wherein:
the passive pressure-controlled mechanical flow regulator includes
i) a first pressure relief valve having an inlet fluidly coupled to the second flowpath and an outlet fluidly coupled to the first flowpath;
ii) a second pressure relief valve having an inlet fluidly coupled to the first flowpath and an outlet fluidly coupled to the third flowpath;
iii) a third pressure relief valve having an inlet fluidly coupled to the third flowpath and an outlet fluidly coupled to the first flowpath; and
iv) a fourth pressure relief valve having an inlet fluidly coupled to the first flowpath and an outlet fluidly coupled to the second flowpath.

6. The device of claim 5, wherein:
the first and third pressure relief valves are configured to open at respective first predefined supply pressures corresponding to a desired rate of infusion; and
the second and fourth pressure relief valves are configured to open at respective second predefined supply pressures greater than the first predefined supply pressures.

7. The device of claim 6, wherein:
the first and second pressure relief valves are configured to deliver a therapeutic agent supplied under pressure by pumping action of the least one manual syringe pump into and through the lumen of the infusion catheter at the desired rate of infusion and direct any excess flow into the least one additional manual syringe pump; and
the third and fourth pressure relief valves are configured to deliver a therapeutic agent supplied under pressure by pumping action of the least one additional manual syringe pump into and through the lumen of the infusion catheter at the desired rate of infusion and direct any excess flow into the least one manual syringe pump.

8. The device of claim 5, wherein:
the connector body further includes a passive flow restrictor downstream of the first, second, third, and fourth pressure relief valves.

9. The device of claim 1, wherein:
the connector body further includes a secondary connector for fluidly coupling and detachably connecting to at least one further manual syringe pump in a configuration that bypasses the passive pressure-controlled mechanical flow regulator.

10. The device of claim 9, wherein:
the connector body further includes a first flowpath that extends through the first connector and is fluidly coupled to the lumen of the infusion catheter during use, a second flowpath that extends through the second connector and is fluidly coupled to the least one manual syringe pump during use, and an additional flowpath that extends through the secondary connector and is fluidly coupled to the least one further manual syringe pump during use, wherein the passive pressure-controlled mechanical flow regulator is fluidly coupled to both the first flowpath and the second flowpath, and the additional flowpath is fluidly coupled to the second flowpath downstream of the passive pressure-controlled mechanical flow regulator.

11. The device of claim 10, wherein:
the connector body further includes a check valve disposed between the passive pressure-controlled mechanical flow regulator and the additional flowpath.

12. The device of claim 9, wherein:
the connector body is configured to deliver a therapeutic agent supplied under pressure by pumping action of the least one manual syringe pump into and through the lumen of the infusion catheter at a desired rate of infusion; and
the connector body is further configured to deliver a secondary fluid supplied under pressure by pumping action of the least one further manual syringe pump into and through the lumen of the infusion catheter.

13. The device of claim 1, wherein:
the passive pressure-controlled mechanical flow regulator comprises a chamber with an inlet port leading into interior space of the chamber and a restrictor tube that extends into the interior space of the chamber, wherein the restrictor tube includes a restrictor inlet disposed within the interior space of the chamber and an annular elastomeric membrane spaced from the restrictor inlet.

14. The device of claim 13, wherein:
the elastomeric membrane is configured to deform or deflect radially inward to regulate fluid flow through the restrictor tube.

15. The device of claim 13, wherein:
the restrictor tube further comprises a bypass valve having an inlet in fluid communication with the interior space of the chamber and an outlet in fluid communication with the lumen of the restrictor tube.

16. The device of claim 15, wherein:
the bypass valve is configured to open at a predefined pressure within the interior space of the chamber that is greater than pressure corresponding to a desired infusion rate of fluid flow through the restrictor tube.

17. The device of claim 1, wherein:
the passive pressure-controlled mechanical flow regulator comprises a restrictor orifice of fixed size that corresponding to a desired infusion rate.

18. The device of claim 17, wherein:
the fixed size is based on infusion of fluid of known viscosity pumped by a manual syringe pump of predefined size within a predefined operating pressure range.

19. A system for treating a patient, comprising:
at least one infusion catheter for treatment of a patient, wherein the at least one infusion catheter has a proximal end with a hub, a distal end with a distal tip, and a lumen extending from the hub to the distal tip through which to infuse a first fluid into the patient; and
at least one connector body having a first connector for fluidly coupling and detachably connecting to the hub of the at least one infusion catheter, a second connector for fluidly coupling and detachably connecting to at least one manual syringe pump containing the first fluid, and a passive pressure-controlled mechanical flow regulator that regulates the flow rate of the first fluid supplied by manual pump action of the least one manual syringe pump to the infusion catheter and delivered by the infusion catheter into the vascular system of the patient.

20. The system of claim 19, wherein:
the passive pressure-controlled mechanical flow regulator is configured to deliver the first fluid supplied under pressure by pumping action of the least one manual syringe pump into and through the lumen of the infusion catheter at a desired rate of infusion.

21. The system of claim 19, wherein:
the connector body further includes a first flowpath that extends through the first connector and is fluidly coupled to the lumen of the infusion catheter during use and a second flowpath that extends through the second connector and is fluidly coupled to the least one manual syringe pump during use, wherein the passive pressure-controlled mechanical flow regulator is fluidly coupled to both the first flowpath and the second flowpath.

22. The system of claim 21, wherein:
the connector body further includes a third connector for fluidly coupling and detachably connecting to at least one additional manual syringe pump and a third flowpath that extends through the third connector and is fluidly coupled to the least one additional manual syringe pump during use, wherein the passive pressure-controlled mechanical flow regulator is fluidly coupled to the third flowpath.

23. The system of claim 22, wherein:
the passive pressure-controlled mechanical flow regulator includes
i) a first pressure relief valve having an inlet fluidly coupled to the second flowpath and an outlet fluidly coupled to the first flowpath;
ii) a second pressure relief valve having an inlet fluidly coupled to the first flowpath and an outlet fluidly coupled to the third flowpath;
iii) a third pressure relief valve having an inlet fluidly coupled to the third flowpath and an outlet fluidly coupled to the first flowpath; and
iv) a fourth pressure relief valve having an inlet fluidly coupled to the first flowpath and an outlet fluidly coupled to the second flowpath.

24. The system of claim 23, wherein:
the connector body further includes a passive flow restrictor downstream of the first, second, third, and fourth pressure relief valves.

25. The system of claim 23, wherein:
the first and third pressure relief valves are configured to open at respective first predefined supply pressures corresponding to a desired rate of infusion; and
the second and fourth pressure relief valves are configured to open at respective second predefined supply pressures greater than the first predefined supply pressures.

26. The system of claim 25, wherein:
the first and second pressure relief valves are configured to deliver the first fluid supplied under pressure by pumping action of the least one manual syringe pump into and through the lumen of the infusion catheter at the desired rate of infusion and direct any excess flow into the least one additional manual syringe pump; and
the third and fourth pressure relief valves are configured to deliver the first fluid supplied under pressure by pumping action of the least one additional manual syringe pump into and through the lumen of the infusion catheter at the desired rate of infusion and direct any excess flow into the least one manual syringe pump.

27. The system of claim 19, wherein:
the connector body further includes a secondary connector for fluidly coupling and detachably connecting to at least one further manual syringe pump in a configuration that bypasses the passive pressure-controlled mechanical flow regulator.

28. The system of claim 27, wherein:
the connector body further includes a first flowpath that extends through the first connector and is fluidly coupled to the lumen of the infusion catheter during use, a second flowpath that extends through the second connector and is fluidly coupled to the least one manual syringe pump during use, and an additional flowpath that extends through the secondary connector and is fluidly coupled to the least one further manual syringe pump during use, wherein the passive pressure-controlled mechanical flow regulator is fluidly coupled to both the first flowpath and the second flowpath, and the additional flowpath is fluidly coupled to the second flowpath downstream of the passive pressure-controlled mechanical flow regulator.

29. The system of claim 28, wherein:
the connector body further includes a check valve disposed between the passive pressure-controlled mechanical flow regulator and the additional flowpath.

30. The system of claim 27, wherein:
the connector body is configured to deliver the first fluid supplied under pressure by pumping action of the least one manual syringe pump into and through the lumen of the infusion catheter at a desired rate of infusion; and
the connector body is further configured to deliver a secondary fluid supplied under pressure by pumping action of the least one further manual syringe pump into and through the lumen of the infusion catheter.

31. The system of claim 19, wherein:
the passive pressure-controlled mechanical flow regulator comprises a chamber with an inlet port leading into interior space of the chamber and a restrictor tube that extends into the interior space of the chamber, wherein the restrictor tube includes a restrictor inlet disposed within the interior space of the chamber and an annular elastomeric membrane spaced from the restrictor inlet.

32. The system of claim 31, wherein:
the elastomeric membrane is configured to deform or deflect radially inward to regulate fluid flow through the restrictor tube.

33. The system of claim 31, wherein:
the restrictor tube further comprises a bypass valve having an inlet in fluid communication with the interior space of the chamber and an outlet in fluid communication with the lumen of the restrictor tube.

34. The system of claim 33, wherein:
the bypass valve is configured to open at a predefined pressure within the interior space of the chamber that is greater than pressure corresponding to a desired infusion rate of fluid flow through the restrictor tube.

35. The system of claim 19, wherein:
the passive pressure-controlled mechanical flow regulator comprises a restrictor orifice of fixed size that corresponding to a desired infusion rate.

36. The system of claim 33, wherein:
the fixed size is based on infusion of fluid of known viscosity pumped by a manual syringe pump of predefined size within a predefined operating pressure range.

37. The system of claim 19, wherein:
the infusion catheter includes an occluder at its distal end adapted to prevent reflux of the first fluid.

38. The system of claim 37, wherein:
the occluder is a dynamic occluder.

39. The system of claim 38, wherein:
the dynamic occlude is a microvalve.

40. The system of claim 19, wherein:
the first fluid is a therapeutic agent or a secondary fluid.

41. A kit for use with at least one infusion catheter for treatment of a patient, wherein the at least one infusion catheter has a proximal end with a hub, a distal end with a distal tip, and a lumen extending from the proximal end to the distal tip, the kit comprising:
a plurality of connector bodies having a first connector for fluidly coupling and detachably connecting to the hub of the at least one infusion catheter, a second connector for fluidly coupling and detachably connecting to at least one manual syringe pump, and a passive pressure-controlled mechanical flow regulator that regulates fluid flow supplied by manual pump action of the least one manual syringe pump to the infusion catheter and delivered by the infusion catheter into the vascular system of the patient, wherein the passive pressure-controlled mechanical flow regulator of the plurality of connector bodies comprise restrictors orifice of varying fixed sizes that corresponding to desired infusion rates for a number of different fluids.

42. The kit of claim 37, wherein:
the varying fixed sizes are based on infusion of different fluids of known viscosity pumped by manual syringe pumps of predefined sizes within different predefined operating pressure ranges.

43. A method of treating a patient, comprising:
providing a system including an infusion catheter and a connector body, the infusion catheter having a proximal end with a hub, a distal end with a distal tip, and a lumen extending from the proximal end to the distal tip, the connector body having a first connector for fluidly coupling and detachably connecting to the hub of the infusion catheter, a second connector for fluidly coupling and detachably connecting to at least one manual syringe pump, and a passive pressure-controlled mechanical flow regulator; and
fluidly coupling and detachably connecting the first connector of the connector body to the hub of the infusion catheter;
fluidly coupling and detachably connecting the second connector of the connector body to a first manual syringe pump; and
manually pumping the first manual syringe pump to pump fluid into the infusion catheter, wherein the passive pressure-controlled mechanical flow regulator of the connector body regulates fluid flow into and through the infusion catheter for delivery by the infusion catheter into the vascular system of the patient.

44. The method of claim 43, wherein:
the connector body is configured to deliver a therapeutic agent into and through the lumen of the infusion catheter at a desired rate of infusion, wherein the therapeutic agent is supplied to the connector body under pressure by the manual pumping of the first manual syringe pump.

45. The method of claim 44, wherein:
the connector body further includes a first flowpath that extends through the first connector and is fluidly coupled to the lumen of the infusion catheter during use and a second flowpath that extends through the second connector and is fluidly coupled to the first manual syringe pump during use, wherein the passive pressure-controlled mechanical flow regulator is fluidly coupled to both the first flowpath and the second flowpath.

46. The method of claim 45, wherein:
the connector body further includes a third connector for fluidly coupling and detachably connecting to at least one additional manual syringe pump and a third flowpath that extends through the third connector and is fluidly coupled to the least one additional manual syringe pump during use, wherein the passive pressure-controlled mechanical flow regulator is fluidly coupled to the third flowpath;
the passive pressure-controlled mechanical flow regulator includes
i) a first pressure relief valve having an inlet fluidly coupled to the second flowpath and an outlet fluidly coupled to the first flowpath;
ii) a second pressure relief valve having an inlet fluidly coupled to the first flowpath and an outlet fluidly coupled to the third flowpath;
iii) a third pressure relief valve having an inlet fluidly coupled to the third flowpath and an outlet fluidly coupled to the first flowpath; and
iv) a fourth pressure relief valve having an inlet fluidly coupled to the first flowpath and an outlet fluidly coupled to the second flowpath; and
the method further includes fluidly coupling and detachably connecting the third connector of the connector body to a second manual syringe pump separate and distinct from the first manual syringe pump.

47. The method of claim 46, wherein:
the first and third pressure relief valves are configured to open at respective first predefined supply pressures corresponding to a desired rate of infusion; and
the second and fourth pressure relief valves are configured to open at respective second predefined supply pressures greater than the first predefined supply pressures.

48. The method of claim 47, wherein:
the first and second pressure relief valves are configured to deliver a therapeutic agent supplied under pressure by manual pumping of the first syringe pump into and through the lumen of the infusion catheter at the desired rate of infusion and direct any excess flow into the second manual syringe pump; and
the third and fourth pressure relief valves are configured to deliver a therapeutic agent supplied under pressure by manual pumping of the second manual syringe pump into and through the lumen of the infusion catheter at the desired rate of infusion and direct any excess flow into the first manual syringe pump.

49. The method of claim 43, wherein:
the connector body further includes a secondary connector for fluidly coupling and detachably connecting to at least one further manual syringe pump in a configuration that bypasses the passive pressure-controlled mechanical flow regulator; and
the method further includes fluidly coupling and detachably connecting the secondary connector of the connector body to a secondary manual syringe pump.

50. The method of claim 49, wherein:
the connector body is configured to deliver a therapeutic agent supplied under pressure by manual pumping of the first manual syringe pump into and through the lumen of the infusion catheter at a desired rate of infusion; and
the connector body is further configured to deliver a secondary fluid supplied under pressure by manual pumping of the secondary manual syringe pump into and through the lumen of the infusion catheter.
